SEQUENCE LISTING (5 sequences)

SEQ ID NO: 1 - DNA, 525 bp, Staphylococcus aureus
atgagaggatcgcatcaccatcaccatcacggatctaaaccacatatcgataattatcttcacgataaagataaagatgaaaggattgaacaatatgataaaaatgtaaaagaacaggcgagtaaggataaaaagcagcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtagaagttctagatggatccgcatgcgagctcggtaccccgggtcgacctgcagccaagcttaattag

SEQ ID NO: 2 - DNA, 525 bp, Staphylococcus aureus
tactctcctagcgtagtggtagtggtagtgcctagatttggtgtatagctattaatagaagtgctatttctatttctactttcctaacttgttatactatttttacattttcttgtccgctcattcctatttttcgtcgttcgatttggagtttaaggctttctatttagctttcaccgtccgatataactttaaggtctacgactataatttcttggtcatataggtcctggtcgttgtggacttgttaatttatctccacattcgaaacgtcttcttttacttagtgatctactagttttataaagttaacgtcctgtgtgaaagtaactggcaggcttgatagttaaatgtttagaatttcgtcggttttttccatcataccacatgaaatttcaaccattactttgtgcattcatattttactgttcatattctctacaattcggatgtctacatcttcaagatctacctaggcgtacgctcgagccatggggcccagctggacgtcggttcgaattaatc

SEQ ID NO: 3 - protein, 174 aa, Staphylococcus aureus
MRGSHHHHHHGSKPHIDNYLHDKDKDERIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDGSACELGTPGRPAAKLN

SEQ ID NO: 4 - DNA, 27 bp, Artificial
  Forward primer
ttttttagatctaaaccacatatcgat

SEQ ID NO: 5 - DNA, 26 bp, Artificial
  Reverse primer
ttttttggatcatctagaacttctac

(19) United States
(12) Patent Application Publication
(10) Pub. No.: US 2007/0110761 A1
(43) Pub. Date: May 17, 2007

(54) Protein purification means

(75) Inventor: Christopher Scott, Belfast (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Fusion Antibodies Limited, Belfast (GB)

(21) Appl. No.: 10/540,415

(22) PCT Filed: Dec. 29, 2003
(86) PCT No.: PCT/GB03/05647

§ 371 Date: Jul. 29, 2005

(30) Foreign Application Priority Data
Dec. 28, 2002 (GB) 0230247.9
Jul. 23, 2003 (GB) 0317218.6
Dec. 30, 2002 (GB) PCTGB02005941

(51) Int. Cl. C07H 21/04; A61K 39/02; C12N 9/48; C12N 15/74; C12N 1/21

(52) U.S. Cl. 424/190.1; 435/212; 435/69.3; 435/471; 435/252.3; 536/23.2

(57) ABSTRACT

Described is a novel purification tag, SNUT, based on the gene product of a sortase gene, in particular the srtA gene of Staphylococcus aureus and uses thereof. Also provided are expression constructs comprising DNA enoding the tag for the production of recombinant polypeptides. The tag can be used in methods of purifying soluble domains of a number of proteins previously not able to be isolated efficiently and methods of inducing and/or enhancing an immune response to an antigen of interest.

The present invention relates to purification means, in particular to means suitable for use in purification of soluble proteins.

### INTRODUCTION

The recombinant production of protein in bacteria, yeast, insect and mammalian cell lines has become a cornerstone of biological research and the biotechnology industry. Classical biochemical and chromatographical purification techniques usually produce inadequate amounts of a target protein to study its roles or actions. Even if enough of the protein can be purified, it usually involves cumbersome amounts of starting material or tissue and many processing steps are taken before reasonable purification can be achieved.

Recombinant expression of the target protein bypasses a lot of these problems. By introducing the target protein's gene template to a cell line or bacterial culture, induced overexpression can result in significant levels of that protein being produced. Large amounts of protein make the purification a lot simpler, but the addition or fusion of purification domains or tags allows for a relatively simple one-step purification using affinity chromatography resins. However, occasionally, due to the varying nature of proteins, the production of soluble protein has remained elusive with known tags unable to purify many proteins. In some cases, production of protein can be a problem due to differences in the machinery of bacterial cells. There is therefore a need for a more versatile tag than is available currently on the market. The provision of such a versatile tag enabling, for example, improved ability to quickly produce and screen soluble protein in bacteria such as E. coli would represent a major step forward in protein biochemistry.

### SUMMARY OF THE INVENTION

The present inventors have developed a novel purification tag based on the gene product of a sortase gene, in particular the srtA gene of Staphylococcus aureus. This tag, known as SNUT [Solubility enhancing Unique Tag] has been found to have exceptional activity, enabling the efficient purification of soluble domains of a number of proteins hitherto not able to be isolated efficiently using conventional purification tags.

Throughout this specification, reference to a SNUT Tag should be understood to mean a tag derived from a sortase gene product.

In a first aspect of the invention, there is provided a purification tag comprising a sortase, e.g srtA, gene product.

In preferred embodiments, the sortase gene product is a gene product of the srtA gene of Staphylococcus aureus.

Also provided is the use of a sortase, e.g srtA, gene product as a purification tag.

Furthermore, according to a third aspect of the invention, there is provided an expression construct for the production of recombinant polypeptides, which construct comprises an expression cassette consisting of the following elements that are operably linked: a) a promoter; b) the coding region of a DNA encoding a sortase, eg srtA gene product as a purification tag sequence; c) a cloning site for receiving the coding region for the recombinant polypeptide to be produced; and d) transcription termination signals.

According to a fourth aspect of the invention, there is provided a method for producing a polypeptide, comprising: a) preparing an expression vector for the polypeptide to be produced by cloning the coding sequence for the polypeptide into the cloning site of an expression construct according to the third aspect of the invention; b) transforming a suitable host cell with the expression construct thus obtained; and c) culturing the host cell under conditions allowing expression of a fusion polypeptide consisting of the amino acid sequence of the purification tag with the amino acid sequence of the polypeptide to be expressed covalently linked thereto; and, optionally, d) isolating the fusion polypeptide from the host cell or the culture medium by means of binding the fusion polypeptide present therein through the amino acid sequence of the purification tag.

The expression construct, herein referred to as pSNUT, may be made by modification of any suitable vector to include the coding region of a DNA encoding a sortase. In preferred embodiments, the expression construct is based on the pQE30 plasmid. A sample of PSNUT was deposited with the National Collections of Industrial and Marine Bacteria Ltd. (NCIMB), 23 St Machar Drive, Aberdeen, Scotland AB24 3RY on 23 Dec. 2002 under accession no NCIMB 41153.

In a fifth aspect, there is provided a fusion polypeptide obtained by the method of the fourth aspect of the invention.

The inventors have found that when a fusion polypeptide comprising a polypeptide/protein of interest and a SNUT tag is used as an antigen, the immune response generated is significantly stronger than that generated when the polypeptide/protein of interest alone is used as the antigen.

Thus, in a sixth aspect of the present invention, there is provided a method of inducing and/or enhancing an immune response to an antigen of interest, the method comprising administering the antigen of interest with a sortase, e.g srtA, gene product. The antigen of interest, which preferably is a polypeptide/protein of interest, may be administered simultaneously, separately or sequentially with the sortase, e.g srtA, gene product. In preferred embodiments, the antigen of interest is linked to the sortase, e.g srtA, gene product, preferably in the form of a fusion polypeptide.

In a seventh aspect of the invention, there is provided the use of a sortase, e.g srtA, gene product as an immunogen. As with the sixth aspect, the sortase, e.g srtA, gene product is preferably administered as a fusion polypeptide comprising the sortase, e.g srtA, gene product and an antigen of interest.

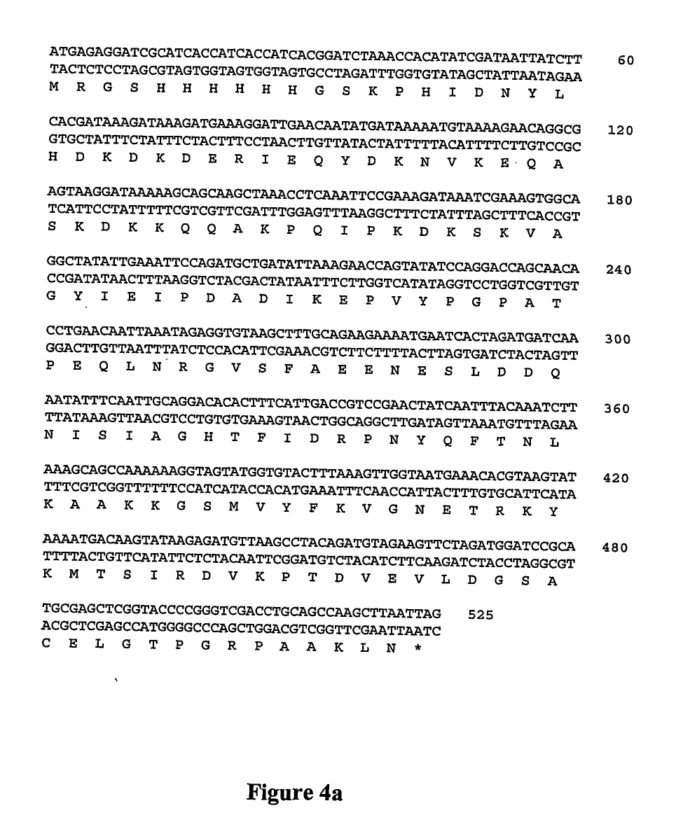

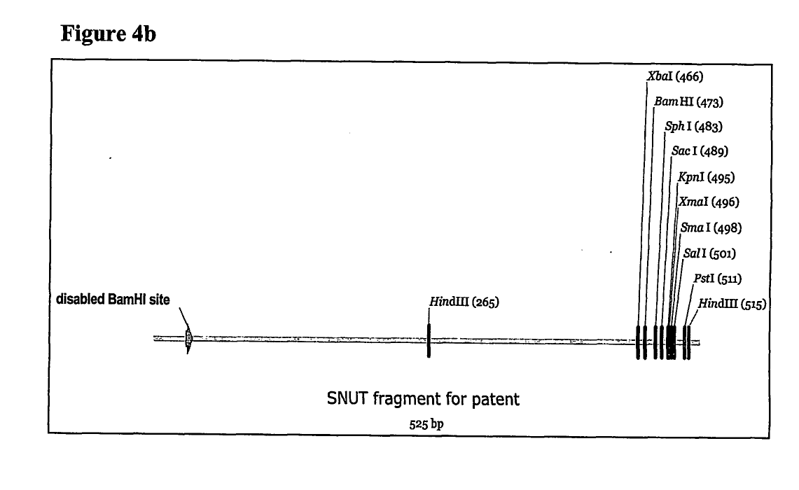

In preferred embodiments, the sortase, e.g. srtA gene product (SNUT) is encoded by the nucleotide sequence shown in FIG. 4 or a variant or fragment thereof. Preferably, the srtA gene product comprises amino acids 26 to 171 of the SrtA sequence shown in FIG. 4 or a variant or fragment thereof.

Variants and fragments of and for use in the invention preferably retain the functional capability of the polypeptide i.e. ability to be used as a purification tag. Such variants and fragments which retain the function of the natural polypeptides can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York.

A variant nucleic acid molecule shares homology with, or is identical to, all or part of the coding sequence discussed above. Generally, variants may encode, or be used to isolate or amplify nucleic acids which encode, polypeptides which are capable of ability to be used as a purification tag.

Variants of the present invention can be artificial nucleic acids (i. e. containing sequences which have not originated naturally) which can be prepared by the skilled person in the light of the present disclosure. Alternatively they may be novel, naturally occurring, nucleic acids, which may be isolatable using the sequences of the present invention. Thus a variant may be a distinctive part or fragment (however produced) corresponding to a portion of the sequence provided in FIG. 4. The fragments may encode particular functional parts of the polypeptide.

The fragments may have utility in probing for, or amplifying, the sequence provided or closely related ones.

Sequence variants which occur naturally may include alleles or other homologues (which may include polymorphisms or mutations at one or more bases). Artificial variants (derivatives) may be prepared by those skilled in the art, for instance by site directed or random mutagenesis, or by direct synthesis. Preferably the variant nucleic acid is generated either directly or indirectly (e. g. via one or amplification or replication steps) from an original nucleic acid having all or part of the sequences of FIG. 4. Preferably it encodes a polypeptide which can be used as a purification tag. The term ‘variant’ nucleic acid as used herein encompasses all of these possibilities. When used in the context of polypeptides or proteins it indicates the encoded expression product of the variant nucleic acid.

Homology (i. e. similarity or identity) may be as defined using sequence comparisons are made using FASTA and FASTP (see Pearson & Lipman, 1988. Methods in Enzymology 183:6398) Parameters are preferably set, using the default matrix, as follows: Gapopen (penalty for the first residue in a gap):−12 for proteins/−16 for DNA Gapext (penalty for additional residues in a gap):−2 for proteins/−4 for DNA KTUP word length: 2 for proteins/6 for DNA. Homology may be at the nucleotide sequence and/or encoded amino acid sequence level. Preferably, the nucleic acid and/or amino acid sequence shares at least about 60%, or 70%, or 80% homology, most preferably at least about 90%, 95%, 96%, 97%, 98% or 99% homology with the sequence shown in FIG. 4.

Thus a variant polypeptide in accordance with the present invention may include within the sequence shown in FIG. 4, a single amino acid change or 2, 3, 4, 5, 6, 7, 8, or 9 changes, or about 10, 15, 20, 30, 40 or 50 changes. In addition to one or more changes within the amino acid sequence shown, a variant polypeptide may include additional amino acids at the C terminus and/or N-terminus.

Naturally, regarding nucleic acid variants, changes to the nucleic acid which make no difference to the encoded polypeptide (i.e. ‘degeneratively equivalent’) are included within the scope of the present invention.

Preferred variants include one or more of the following changes (using the annotation of AF162687): nucleotide 604 AΔG causing an amino acid mutation of KΔR; nucleotide 647 AΔG, codon remains K, therefore a silent mutation; nucleotide 982 GΔA causing an amino acid mutation of GΔE.

Changes to a sequence, to produce a derivative, may be by one or more of addition, insertion, deletion or substitution of one or more nucleotides in the nucleic acid, leading to the addition, insertion, deletion or substitution of one or more amino acids in the encoded polypeptide. Changes may be by way of conservative variation, i. e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation.

Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e. g. altered stability or specificity.

SNUT tags and vectors may be used in methods of purifying a soluble domain of a peptide. Accordingly in a further aspect of the invention, there is provided a method of producing a soluble bioactive domain of a protein, the method comprising the steps of cloning DNA encoding at least one candidate soluble domain into at least one expression vector, transfecting or transforming a host cell with said vector, expressing said DNA in said host cell, wherein said vector encodes a sortase gene product.

The sortase gene product is preferably in the form of a fusion protein.

The method may comprise the steps of analysis of DNA coding for the protein of interest to identify antigenic soluble domains, designing oligonucleotide primers to amplify DNA encoding the domain, amplifying DNA, cloning the DNA, optionally screening clones for correct orientation of DNA, expressing DNA in expression strains, analysing expression products for solubility, analysing products and production of soluble bioactive protein domain.

The method optionally comprises the step of producing a soluble bioactive protein domain of said protein of interest.

The methods and tags of the invention may be used with any suitable polypeptide/protein of interest, for example for the purification of such polypeptides/proteins of interest. As described herein and exemplified in the following examples, the inventors have demonstrated that the methods and tags of the invention enable the efficient purification of a a large number of proteins, many of which have not been amenable to efficient isolation using conventional methods and tags.

In preferred embodiments of the invention, the polypeptide/protein of interest is MAR1, Jak1 or CD33, or a fragment thereof.

In particularly preferred embodiments, the polypeptide/protein of interest is a variable domain fragment e.g. a variable domain fragment of CD33.

Preferred features of each aspect of the invention are as for each of the other aspects mutatis mutandis.

The invention is exemplified with reference to the following non limiting description and the accompanying figures in which:

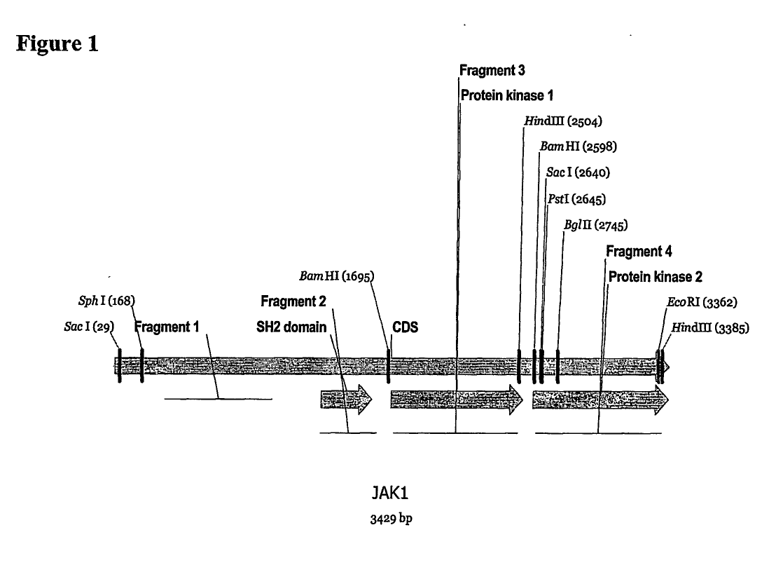

FIG. 1 shows selected domains for amplification from in silico analysis. Representation of a candidate protein for the expression platform, in this case Jak1 (human). Four fragments have been chosen by analysis as depicted.

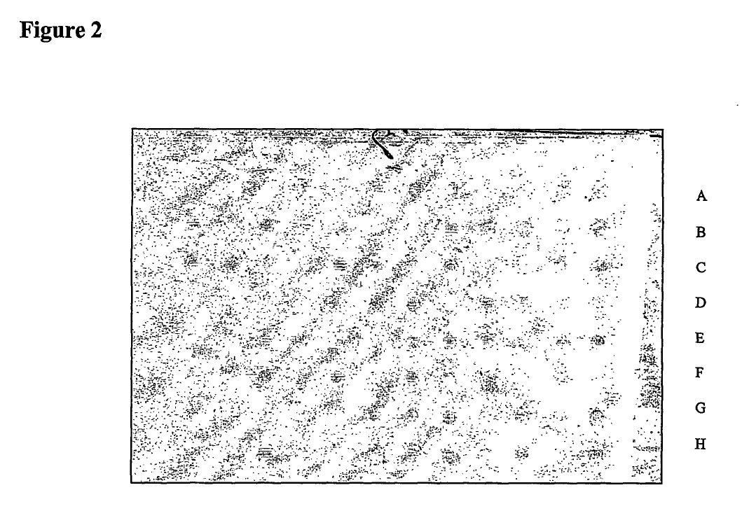

FIG. 2 shows denaturing dot-blot analysis of expression clones of fragments of MAR1 in pQE30.

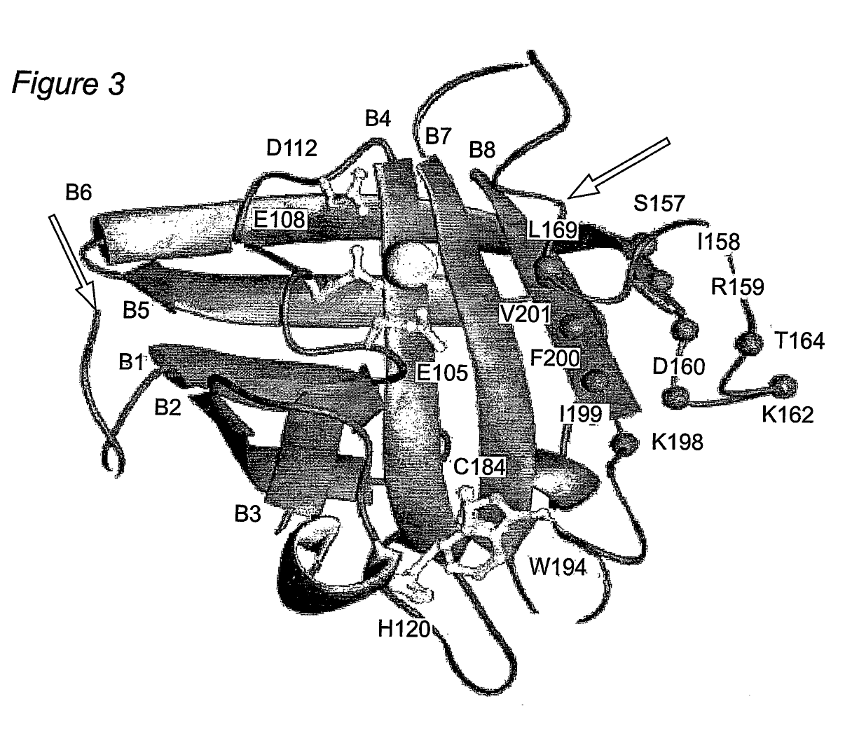

FIG. 3 shows a ribbon Diagram of Staphylcoccus aureus sortase. Ribbon diagram of the putative structure of S. aureus SrtA protein (minus its N-terminal membrane anchor). SNUT represents the portion of this structure between the two yellow arrows as shown. The yellow ball signifies a Ca²⁺ ion, essential for the biological activity of this protein. This diagram is taken from IIangovan et al., 2001 , PNAS 98 (11) 6056 (doi:10.1073/pnas.101064198)

FIG. 4 shows the Nucleotide Sequence and amino acid sequence of SNUT fragment.

(a) This is the determined sequence of SNUT. The fragment was cloned into pQE30 using the BamHI site of this vector. When in the wanted orientation, insertion results in the inactivation of the upstream cloning site, therefore allowing any subsequent cloning of target inserts with the downstream BamHI site (see (b) for restriction map of sequence).

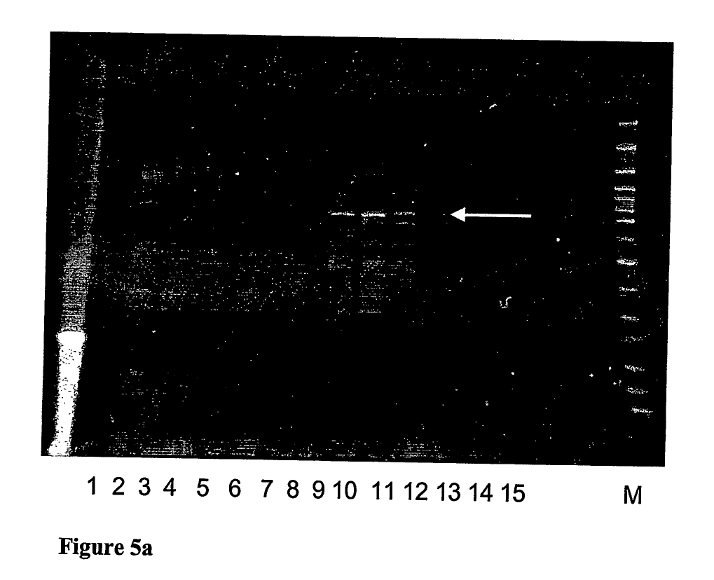

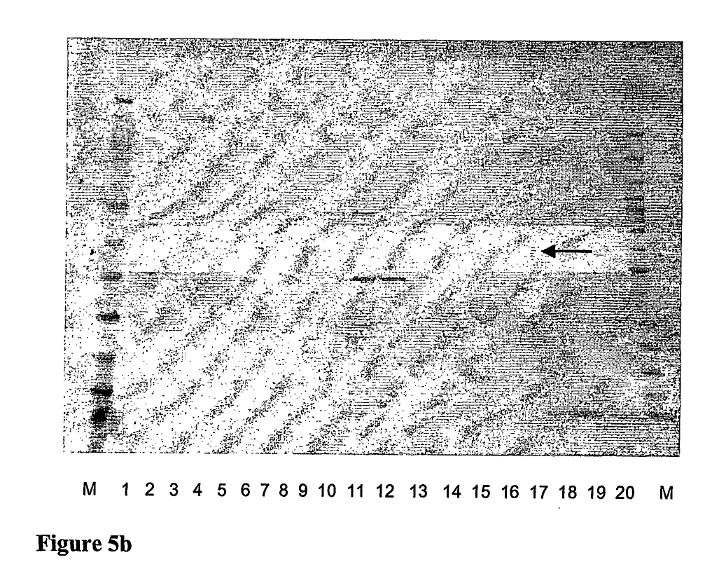

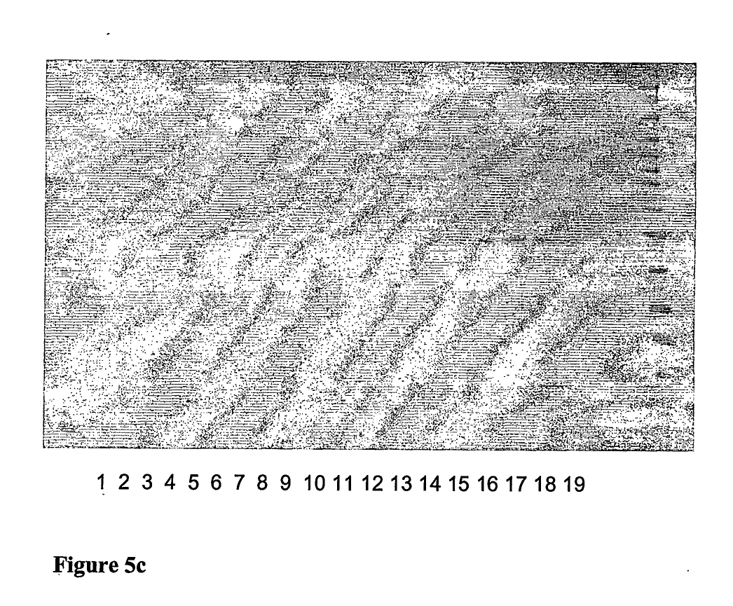

FIG. 5 illustrates qualitative purification results using the SNUT fusion tag. (a) shows the elution profile on SDS-PAGE of SNUT-Jak1 using AKTA Prime native histag purification. Successful elution of SNUT-Jak1 construct is signified by the white arrow. (b) shows the elution profile on SDS-PAGE of SNUT-MAR1 using AKTA Prime native histag purification. Successful elution is shown by the arrow. (c) shows the same gel stained in (b) western blotted and detected using poly-histidine-HRP antibody. This is confirmation that the eluted species in (b) is actually SNUT-MAR1, of expected molecular weight.

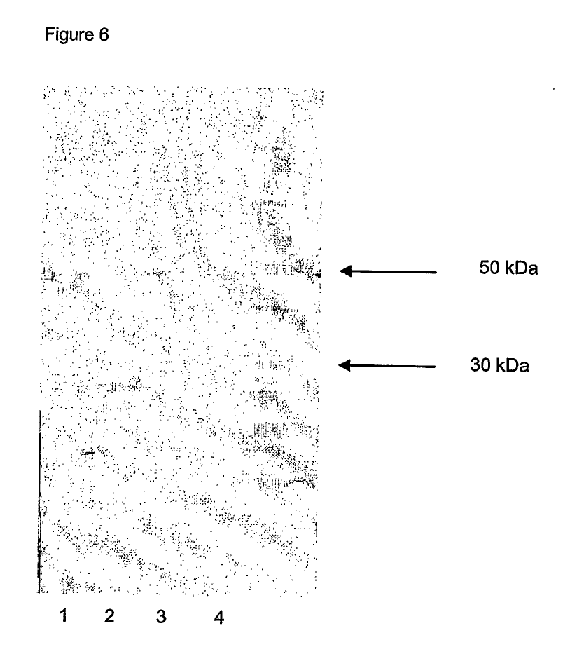

FIG. 6 shows a Western blot of lysates using anti-histag antibody.

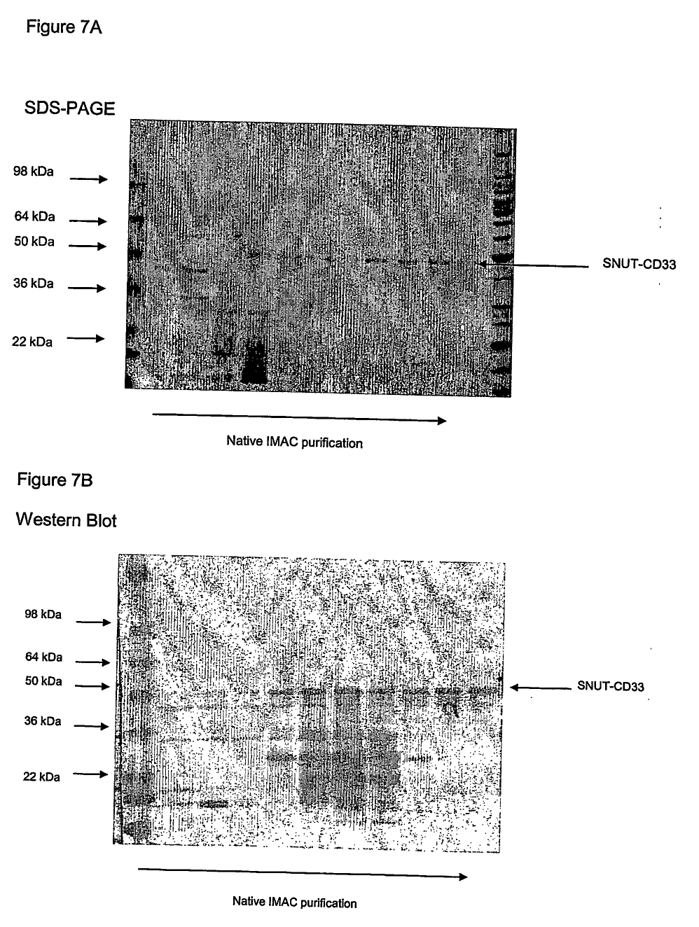

FIG. 7a illustrates the elution profile on SDS-PAGE of SNUT-CD33.

FIG. 7b illustrates a Western blot of the same gel from FIG. 7a using anti-histag antibody to detect the proteins.

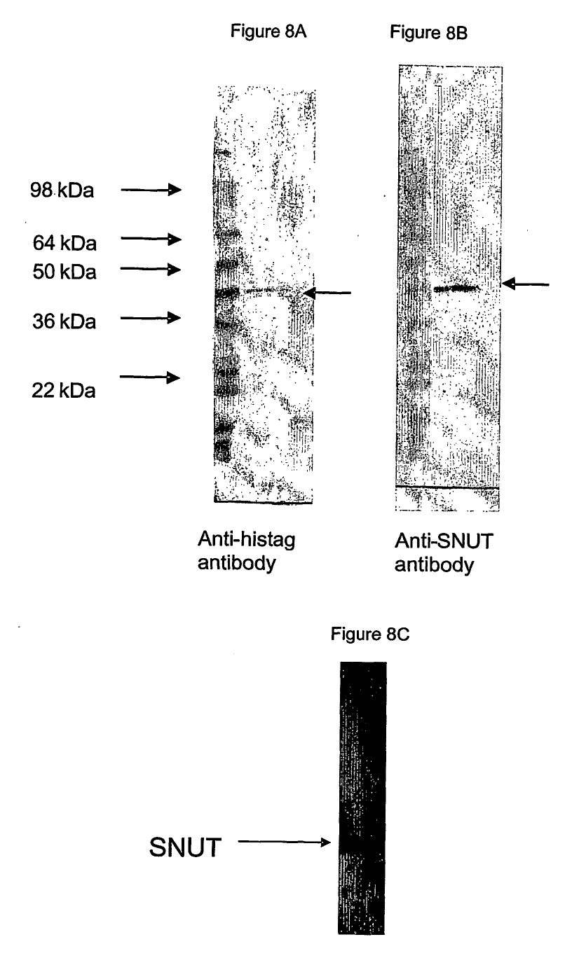

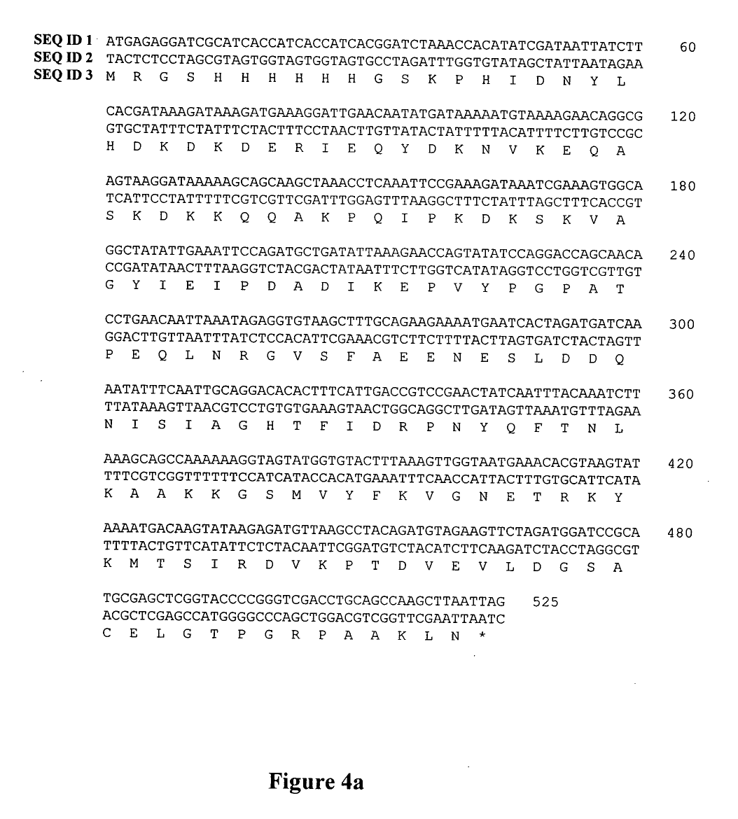

FIG. 8a illustrates a Western blot using anti-histag antibody to detect the proteins.

FIG. 8b illustrates a Western blot of the same gel as FIG. 8b using anti-SrtA antibody to detect the proteins.

FIG. 8C shows a Western blot showing the detection of the SNUT protein using an anti-SrtA monoclonal antibody.

### TEMPLATE ANALYSIS AND PRIMER DESIGN

Analysis of the DNA coding for a protein of interest may be performed using software packages such as Vector NTI (Informax, USA) and BLASTP(http://www.ncbi.nlm.nih.gov/BLAST/), p-fam (www.sanger.ac.uk/pfam) and TM pred (www.hgmp.mrc.ac.uk) which may be used to identify complete domains within the protein that significantly increase the likelihood of antigenicity and/or solubility when expressed as a subunit of the original protein coding sequence.

In order to increase the possibility of identifying a soluble domain, preferably multiple sub-domains, more preferably at least three sub-domains, for example 3 to 9 sub-domains may be identified for processing.

Oligonucleotide primers to amplify the selected sub-domains may be designed with the help of commercially avialable software packages such as the internet software package Primer3 (http://www-genome.wi.mit.edu/genome software/other/primer3.html (Whitehead Institute for Biomedical Research), Vector NTI (www.informaxinc.com) and DNASIS (Hitachi Software Engineering Company (www.oligo.net).

Typically primers for use in a method of the invention are in the range 10-50 base pairs in length, preferably 15 to 30, for example 20 base pairs in length, with annealing temperatures in the range 45-72° C., more conveniently 55-60° C. Primers may be synthesised using standard techniques or may be sourced from commercial suppliers such as Invitrogen Life Technologies (Scotland) or MWG-Biotech AG (Germany).

PCR of Insert

The desired inserts which encode the selected sub-domains are amplified using the primers designed specifically for that target gene using standard PCR techniques. The template DNA for amplification can be in the form of plasmid DNA, CDNA or genomic DNA, depending on whatever is appropriate or indeed available. Any suitable DNA polymerase may be used, for example, Platinum Taq, Pfu (www.stratagene.com) or Pfx (www.invitrogen.com). Any suitable PCR system may be used, for example, the Expand High Fidelity PCR system (Roche, Basel, Switzerland).

Several different thermocycler conditions may be used with each set of primers. This increases the chance of the PCR working without having to individually optimise each new primer set. Typically the following three programs may be used in the method:
1. A standard PCR programme using the recommended annealing temperature provided with the primers.
2. A standard PCR programme using 50° C. as the temperature for annealing.
3. A touchdown PCR programme, where the annealing temperature starts at a high temperature e.g 65° C. for 10 cycles and then gradually decreases the annealing temperature to 50° C. over the subsequent e.g 15 cycles.

Buffer conditions may be adjusted as required, for example with respect to magnesium ion concentration or addition of DMSO for the amplification of difficult templates. Further details of a suitable purification method which may be used with the vector or tag of the invention can be found in our co-pending PCT application PCT/GB02/05941, filed on the same day as this application, published 24 Jul. 2003, and claiming priority from GB 0131026.7.

The PCR products may be visualised using standard techniques, for example on a 1.5% agarose gel stained with Ethidium Bromide and the bands are cut out of the gel and purified using Mini elute gel extraction Kit (Qiagen, Crawley, England).

Expression Vectors

Amplified DNA inserts may be cloned into expression vectors using techniques dictated by the multiple cloning sites of the vector in question. Such techniques are readily available to the skilled person.

Any suitable expression system can be used in the invention. Preferably, the expression system is prokaryotic. Suitable vectors for use in the method of the invention include any vector which can encode SNUT [Solubility enhancing Unique Tag], for example pSNUT. This tag is based on the sequence of a trans-peptidase found on the surface of gram-positive bacteria. This protein is highly soluble, and expressed as very high levels.

The inventors have found that SNUT is an ideal fusion tag for conferring solubility and expression levels to target protein fragments. SNUT may be cloned into any suitable vector. For the purposes of the examples shown in this application, the sequence incorporating the SNUT fragment is cloned into pQE30 (Qiagen, Valencia, Calif.) in a manner allowing full use of the multiple cloning site (MCS) of this vector for downstream gene insertions.

Development of pSNUT

The inventors found that a tag based on the srtA gene product from Staphylcoccus aureus is highly soluble, reacts well to purification schemes and expresses particularly well. It was hypothesised that the incorporation of a portion or domain of this protein could represent a useful fusion tag in the present method, and indeed the expression of any poorly soluble protein in E. coli. Using NMR studies, the 3D structure of this protein has been predicted and is shown in FIG. 3. We hypothesised that by taking a portion of this structure, we could make a manipulateable protein tag, but not disturb its tertiary structure enough to reduce its highly favourable characteristics listed above. The region of this protein used as a solubility-enhancing tag is depicted by two arrows.

The SNUT tag was cloned into pQE30. However, it may be cloned into any suitable expression vector. Positive clones may be identified by denaturing dot blots, SDS-PAGE and Western blotting. Final confirmation of these clones was provided by DNA sequencing, and the sequence of the multiple cloning region of the resultant vector is shown in FIG. 4.

Variances in the sequence of the SNUT domain were observed from the sequence for SrtA that has been logged in Genbank (AF162687). The variances are (using the annotation of AF162687) nucleotide 604 AΔG causing an amino acid mutation of KAR; nucleotide 647 AΔG, codon remains K, therefore a silent mutation; nucleotide 982 GΔA causing an amino acid mutation of GΔE.

Preliminary trials and native purification showed that the SNUT fragment was very soluble and its characteristics were in no way diminished by truncation, thus showing that SNUT could represent a useful tag domain (data not shown). As described in the Examples, to fully test the abilities of SNUT, we then chose two proteins were soluble protein production had proved impossible using conventional methods and using the other expression systems of the method of the present invention. Surprisingly, we found that, using pSNUT in the method of the invention, these proteins could be produced in soluble form.

Clone Propagation

Target insert/expression vector ligations may be propagated using standard transformation techniques including the use of chemically competent cells or electro-competent cells. The choice of the host cell and strain for transformation is dependent on the characteristics of the expression vectors being utilised.

Bacterial cells, for example, Escherichia coli, are the preferred host cells. However, any suitable host cell may be used. In preferred embodiments, the host cells are Escherchia coli.

The vectors may be used to each transfect or transform a plurality of different host cell strains. The set of host cell strains for individual vector may be the same or different from the set used with other vectors.

In a particularly preferred embodiment of the invention, each vector may be transformed into three E. coli strains (for example, selected from Rosetta(DE3)pLacI, Tuner(DE3)pLacI, Origami BL21 (DE3)pLacI and TOP10F, Qiagen).

Where the vectors are pQE based vectors, TOP10F′ cells are preferred for the propagation and expression trials of such vectors. The present inventors have identified this strain as a more superior strain for these vectors than either of the recommended strains by the supplier (M15 and SG13009), in terms of ease of use and culture maintenance (only one antibiotic required as to two with M15 or SG13009 (www.quiagen.com). Other F′ strains such as XL1 Blue can be used, but are inferior to the TOP10F′ strain, due to lack of expression regulation (results not shown). The use of TOP10F′ (Invitrogen) for the propagation and/or expression pQE based vectors forms an independent aspect of the present invention. Other F′ strains such as XL1 Blue may also be used, but are inferior to the TOP10F′.

After transformation, cells may be plated out onto selection plates and propagated for the development of single colonies using standard conditions.

Propagation of Cells

The colonies may be used to inoculate duplicate wells in a 96 well plate.

Typically, each well may contain 200 μl of LB broth with the appropriate antibiotics. Each plate may be dedicated to one strain of E. coli or other host cell which alleviates the problems of different growth rates. The necessary controls are also included on each plate. The plates are then grown up, preferably at 37° C. or any other temperature as appropriate to the particular host cell and vector, with shaking, until log phase is reached. This is the primary plate.

From the primary plate a secondary plate is seeded and then grown. Typically, the secondary plate is be seeded using ‘hedgehog’ replicators and then grown up to, for example, log phase, chilled to 16° C. for 1 hour. Determination of positive clones from these plates may be undertaken using functional studies. Routinely, 6-48 clones for each insert-vector ligation are taken and propagated in culture micro-titre plates containing up to 500 μl of media. According to the conditions and reagents required, protein production is then induced, and cultures propagated further. Most vectors are under the control of a promoter such as T7, T7lac or T5, and can be easily induced with IPTG during log phase growth. Typically, cultures are propagated in a peptone-based media such as LB or 2YT supplemented with the relevant antibiotic selection marker. These cultures are grown at temperatures ranging from 4-40° C., but more frequently in the range of 20-37° C. depending on the nature of the expressed protein, with or without shaking and induced when appropriate with the inducing agent (usually log or early stationary phase). After induction, growth propagation can be continued for 1-16 hours for a detectable amount of protein to be produced.

The primary plate is preferably stored at 4° C. until the process is complete.

Colony Screening for Inserts in Correct Orientation

The method of the invention may include the step of testing transformants for correct orientation of the inserts. Identification of positive clones can be achieved through a variety of methods, including standard techniques such as digestion analysis of plasmid DNA; colony PCR and DNA sequencing. Alternatively, dot-blotting may be used for the identification of positive clones for example, using a BioDot apparatus (BioRad) containing nitrocellulose membrane (0.45 μM pore size) in accordance with the manufacturers' instructions, prior to final confirmation by DNA sequencing.

The use of this dot blotting method in the platform represents a rapid, reproducible and robust detection method. This particular method is useful for the rapid detection or presence of recombinant protein and allows for a determination of all clones irrespective of solubility and conformation. This may be important at this stage, because conformational structures can inhibit the detection of tag domains if they are not presented properly on the surface of the protein. This can occur as easily with both soluble and insoluble protein.

As described above, standard colony PCR techniques may be used. For example, transformants may be selected, either manually or using automation such as the Cambridge BioRobitics BioPick instrument, and screened using directional PCR using a primer that encodes for a sequence on the vector such as S Tag or GATA sequence, and then the complementary primer from the insert. A PCR mix may be used such as the RedTaq DNA Polymerase (Sigma Aldrich, Dorset, England) and the thermocycler conditions used may be the standard PCR programme using 50° C. as the annealing temperature or adjusted as required.

Although all colony selecting and picking can be done manually, automated colony pickers are preferred. Automated colony pickers such as the BioRobotics BioPick allow for the uniform and reproducible selection of clones from transformation plates. Clone selection determinants can be set to ensure picking colonies of a standardised size and shape. After picking and plate inoculation, propagation of clones can be carried out as described above.

Identification of positive clones can be achieved through a variety of methods, including standard techniques such as digestion analysis of plasmid DNA; colony PCR and DNA sequencing Alternatively, in a preferred embodiment, the novel method of dot-blotting described herein for the identification of positive clones may be used in place of such traditional techniques, prior to final confirmation by DNA sequencing. The use of this method in the platform presented here is not essential in the use of this platform over existing screening methodologies, but represents a rapid, reproducible and robust detection method. The protocol described here is a new protocol for an existing method for which commercially available equipment (Bio-Rad DotBlot) can be purchased.

This particular method is useful for the rapid detection or presence of recombinant protein and allows for a determination of all clones irrespective of solubility and conformation. This is useful at this stage, because conformational structures can inhibit the detection of tag domains if they are not presented properly on the surface of the protein. This can occur as easily with both soluble and insoluble protein.

For example, after growth on the micro-titre plates is complete, the plate is centrifuged at 4000 rpm for 10 minutes at 4° C. to harvest the bacterial cells. The supernatant is removed and the cell pellets are re-suspended in 50 μl lysis buffer (10 mM Tris.HCl, pH 9.0, 1 mM EDTA, 6 mM MgCl₂) containing benzonase (1 μl/ml). The plate is subsequently incubated at 4° C. with shaking for 30 minutes. A sample (10 μl) of the cell lysate is added to 100 μl buffer (8 M urea, 500 mM NaCl, 20 mM sodium phosphate, pH 8.0) and incubated at room temperature for 20 minutes. Samples are then applied to a BioDot apparatus (BioRad) containing nitrocellulose membrane (0.45 μM pore size) in accordance with the manufacturers' instructions. The membrane is removed and transferred into blocking reagent (3% w/v; Bovine serum albumin in TBS) for 30 minutes at room temperature. The blot is washed briefly with TBS then incubated in a primary antibody, specific to the tag being used for the subset of expression clones. Depending on the nature of the primary i.e., whether or not it has a horse radish peroxidase (HRP) reporter function, will depend on whether the use of a secondary is required. For detection of specific binding the membrane is then washed 2×5 minutes in TBS followed by 1×5 minute wash in 10 mM Tris.HCl pH7.6. Detection of specifically bound antibody is disclosed by the addition of chromogenic substrate (6 mg diaminobenzidine in 10 ml 10 mM Tris.HCl pH 7.6 containing 50 μl 6% H₂O₂). The reaction is stopped by thorough rinsing in water. Positive clones identified by this procedure can then be confirmed by DNA sequencing of the expression construct using now industry-standard techniques and equipment such as ABI and Amersham Biosciences.

Sequencing

The sequencing reactions may be performed using techniques common in the art using any suitable apparatus. For example, sequencing may be performed on the cloned inserts, using the Big Dye Terminator cycle sequencing kits (Applied Biosystems, Warrington, UK) and the specific sequencing primer run on a Peltier Thermal cycler model PTC225 (MJ Research Cambridge, Mass.). The reactions may be run on Applied Biosystems—Hitachi 3310 Sequencer according to the manufacturer's instructions. These sequences are checked to ensure that no PCR generated errors have occurred.

Assessment of Solubility of Positive Clones

The cells of positive clones may be harvested and soluble and insoluble protein detected.

Any suitable techniques known in the art can be used to separate soluble and insoluble protein, such as the use of centrifugation, magnetic bead technologies and vacuum manifold filtrations. Typically, however, the separated proteins are ultimately analysed by acrylamide gel and western blotting. This confirms the presence of recombinant protein at the correct size.

In one embodiment, contents of each well in the 96 well plate are transferred into a Millipore 0.65 μm multi-screen plate. The plate is placed on a vacuum manifold and a vacuum is applied. This draws off the culture medium to waste. The cells are then washed with PBS (optional), again the vacuum is applied to remove the PBS. The multi-screen plate is removed from the manifold and bacterial cell lysis buffer (containing DNAse) (50 μl) is added to each well. The plate is incubated at room temperature for 30 minutes with shaking to facilitate lysis of the cells. A fresh 96 well microtitre plate (ELISA grade) is placed inside the vacuum manifold and the multi-screen plate is placed above it. When a vacuum is applied the contents of each well are drawn into the micro-titre plate below. The vacuum only needs to be applied for 20 seconds. The collected lysate contains the soluble fraction of expressed protein. A sample of the collected lysate may subsequently analysed by SDS-PAGE and Western blotting to confirm both the presence and correct molecular weight of the target protein.

The use of SDS-PAGE and Western blotting can be expensive and time consuming, especially when numerous samples must be analysed for each construct. In light of this we have developed a protocol whereby one gel can be used for both total protein staining and western blotting. This represents a significant improvement in this methodology and obviously allows cost saving, and precise comparisons can be made with regard to total protein and western blotting as both sets of results come from the one gel.

The basis of this protocol is in the ability to use chloroform and UV light to stain protein on an SDS-PAGE gel (Kazmin et al., Anal Biochem, 2001, 301(1) 91-6; doi:10.1006/abio.2001.5488). We have used this technique to great effect as it allows for the extremely rapid staining of a SDS-PAGE gel in less than a tenth of the time taken using other more traditional staining methods such as Commassie Brilliant Blue and Collodial Blue stains. We then decided to take this observation a step further and analyse the ability of a chloroform-stained gel to be used in Western blotting. This would not be expected to work as other stained gels result in the fixing of the protein to the gel and subsequent inability to transfer the protein during blotting. This expectation is coupled to the fact that chloroform is not compatible with western blotting equipment (Bio-Rad SD blotter user's manual). However, fortuitously, we have discovered that with a wash of the chloroform-stained gel in double-distilled water, to remove excess chloroform, and after subsequent soaking in transfer buffer, proteins were effectively transferred during western blotting in contrast to expectations. This transfer was no-less effective than from a gel that has not been pre-stained with chloroform and UV light. FIG. 6 primarily shows results relating to the production of soluble protein by the platform, but also shows the ability to use the chloroform-stained SDS-PAGE derived western blot for the identification of proteins, without any apparent damage caused to the proteins.

The use of a chloroform-stained SDS-PAGE derived western blot for the identification of proteins forms another aspect of the present invention.

Scale-Up and Purification

This analysis provides a picture of the expression status of the clones on each plate. Using this analysis, positive soluble protein expressing clones can be identified for the production of soluble recombinant protein for a given target protein. The clones may be selected and their growth scaled up e.g. to 5 ml scale, using the saved primary plate as an inoculum. Parameters that may be taken into consideration in deciding on the appropriate culture to select for scale-up include the desirability of specific regions for the production of an antigen, the overall expression levels of the clone and factors that may affect affinity purification such as amino acid composition.

### EXAMPLES

### Example 1

### Expression Construct Design

FIG. 1 is a diagrammatic representation of the protein Jak1. Using pfam, the position of distinct domains was established. Further analysis of these domains was then carried out using Tmpred and the Kyle and Dolittle hydrophobicity algorithm to determine the usefulness of these domains as soluble antigens. From this tentative analysis, four domains were selected for amplification and expression analysis. Based on this preliminary in silico analysis, primers specific for a target protein were designed and used to amplify domains selected for analysis.

Vectors (500 ng) were restricted with BamHI (20 units) and SalI (20 units) in the presence of calf intestinal alkaline phosphatase (CIP) (2 units), gel purified and quantified using standard methods. Purified PCR fragments (100 ng) were restricted with BamHI (5 units) and SalI 5 units), gel purified, quantified, and then used in a ligation reaction with the restricted vector again using standard T4 DNA ligase methods (Ready-to-Go T4 DNA ligase, Amersham Biosciences). A sample of the ligation reaction (1 μl) was then used to transform the appropriate competent bacterial cells (TOP10F′ were used here for the pQE based vectors, a modification of the manufacturers recommendations; BL21(DE3)pLysE for pET43.1 a and TOP10F′ for pGEX-Fus). Transformants were selected on LB/ampicillin (100 μg/ml) overnight at 28° C.

A Cambridge BioRobitics BioPick instrument was used for the picking of 24 colonies from each of the transformant plates into flat-bottomed and lidded micro-titre plates. The clones were used to inoculate 150 μl of LB (containing 100 μg/ml ampicillin), and these were allowed to grow overnight at 37° C.

A secondary plate was prepared by the inoculation of 200 μl of LB containing the required supplements with 10 μl of the overnight primary culture. These were then grown at 37° C. Once an optical density (OD) of 0.25 at A550 was reached, IPTG (final concentration, 1 mM) was added to induce expression of the recombinant protein. Culture propagation was continued for another 4 hours prior to harvesting of bacterial cells.

After clones expressing specific recombinant protein have been identified, the solubility of these proteins has to be established prior to clone selection for purification. This can be performed a number of ways including the use of centrifugation and automation-friendly vacuum manifold separations. The results here were obtained using methodologies based around the use of vacuum-assisted filtration to separate soluble and insoluble protein. The filtrates that were produced from the method described were then analysed by SDS-PAGE and Western blotting to confirm the production of a recombinant protein of the correct anticipated molecular weight.

### Example 2

### Design and Construction of SNUT Expression Tag

Based on analysis of the amino acid sequence and predicted structure of SrtA_{ΔN}, it was decided to amplify the region of amino acids 26 to 171 of the SrtA sequence. Amplification was conducted using the forward primer 5′ TTTTTTAGATCTAAACCACATATCGAT and the reverse primer 5′ TTTTTTGGATCCATCTAGAACTTCTAC. This product was then digested with BglI and BamHI and ligated into pQE30 vector which had also been digested with BamHI to form the pSNUT vector. The ligation mix was transformed into TOP10F′ cells and single colonies propagated on LB agar containing 100 μg/ml ampicillin. Clones with the srtA fragment in the correct orientation were screened by expression analysis and positive clones identified using the denaturing dot-blot assay described earlier.

The sequence encoding the SNUT tag was cloned into pQE30 as described earlier and positive clones identified by denaturing dot blots, SDS-PAGE and Western blotting. Final confirmation of these clones was provided by DNA sequencing, and the sequence of the multiple cloning region of the resultant vector is shown in FIG. 4. Variances in the sequence of the SNUT domain were observed from the sequence for SrtA that has been logged in Genbank (AF162687). The variances are (using the annotation of AF162687) nucleotide 604 AΔG causing an amino acid mutation of KΔR; nucleotide 647 AΔG, codon remains K, therefore a silent mutation; nucleotide 982 GΔA causing an amino acid mutation of 15 GΔE.

Example 3

### Trials of SNUT Expression Constructs

Target inserts were cloned into the pSNUT vector using primer construction and digestion of resulting. PCR amplifications with BamHI and SAlI as described earlier. pSNUT was digested with BamHI in a similar manner and the target inserts cloned as described. Clones were screened using the denaturing dot-blot system and then analysed with SDS-PAGE and western blotting. Positive clones were used for preparative 200 ml LB cultures containing 100 μg/ml ampicillin and induced as described earlier. This was grown to an optical density of 0. 5 at A₅₅₀ at 37 OC. Expression of SNUT was then induced with the addition of IPTG (final concentration, 1 mM) and left to grow for another 4 hours. Cells were then harvested by centrifugation at 5K rpm for 15 minutes. Cells were re-suspended in 30 ml PBS containing 0.1% Igepal and lysis induced by two freeze-thaw cycles. The suspension was then sonicated and centrifuged at 5K rpm for 15 minutes. The soluble supernatant was transferred to a fresh container and filtered through a 0.8 μm disc filter to remove final cell debris. This solution was then applied to a Ni²⁺ charged IMAC column (Amersham Biosciences HiTrap Chelating column, 1 ml) using an AKTA Prime low pressure chromatography system and column was then treated using a standard native his-tag purification protocol involving washing of column with 20 mM sodium dihydrogen phosphate pH 8.0 containing 10 mM imidazole, 500 mM NaCl, and elution of soluble his-tagged proteins using 20 mM sodium dihydrogen phosphate pH 8.0 containing 500 mM imidazole, 500 mM NaCl. Elution fractions were then analysed on an SDS-PAGE gel (4-20% SDS-PAGE Bio-Rad Criterion gel), which was stained with chloroform as described earlier. This gel was then subsequently western blotted and the his-tagged protein detected with anti-poly-histidine monoclonal antibody using the techniques described herein.

Preliminary trials and native purification showed that the SNUT fragment was very soluble and its characteristics were in no way diminished by truncation, thus showing that SNUT could represent a useful tag domain (data not shown). To fully test the abilities of SNUT, we then chose two proteins for which soluble protein production had proved impossible using the other expression systems in which SNUT was not used as a tag. These were murine MAR1 and human Jak1. Clones were prepared and selected using the method as described in the Examples above and positive clones were subsequently grown and induced at 37° C. These were then treated to identical native histag purifications. Both proteins behaved very favourably under standard purification conditions as can be seen from the purification profiles in FIG. 5. For both these trial proteins, this was the first example of such purification under soluble conditions. The production of these proteins using conventional techniques has failed to produce any soluble protein, irrespective of expression system or growth conditions used (data not shown). However, as described in this example, when the protein fragments were expressed in pSNUT, soluble proteins can be surprisingly obtained.

The effectiveness of SNUT as a fusion protein is even more significant when it is considered that no special growth conditions were required for the generation of soluble protein. This is remarkable when one considers the protein expressionist's standard GST tag which is not even soluble itself when expressed at 37° C.; 28° C. is required before even the generation of GST on its own without any target protein is observed.

### Example 4

### Purification of CD33 Fragments Using SNUT Expression Constructs

Cloning Results

CD33 contains two extracellular immunoglobulin domains. The extracellular region of the CD33 DNA sequence had been cloned into several vectors for expression, including expression as a fusion tag to DHFR and NusA. None of these vectors produced recombinant CD33 protein. The CD33 extracellular region was also cloned into pSNUT. Both pSNUT and CD33 were restricted with BamHI and HindIII under standard conditions and ligated together using T4 DNA ligase, again under standard manufacturer's protocols. TOP10F′ cells were transformed with the ligation product.

6 colonies were picked from the transformation plate and grown in 150 μl LB in a 96-well plate at 37° C. overnight

Expression Analysis:

The overnight cultures were used to inoculate fresh LB cultures (10 μl into 190 μl LB+50 μg/ml ampicillin) and grown at 37° C. for 2 hours. Expression of the SNUT-CD33 construct was induced with 1 mM IPTG.

Cells were pelleted after 4 hours and lysed in PBS+0.1% Igepal. Lysates were analysed by western blot using anti-histag antibody. As shown in FIG. 6, it was clear that colonies 1, 3 and 4 were positive and 2 was not (SNUT only).

Large Scale Expression:

The clone pertaining to lane 1 of FIG. 6 was chosen for sequencing analysis, which proved successful insertion into the pSNUT vector. This clone was grown in large scale (200 ml) for expression of the SNUT-CD33 construct at 37° C. Expression was induced whenever the OD600=0.4-0.6. After 4-6 hours expression, the cells were pelleted and lysed in 8M urea buffer. Lysates were clarified and purifed by immobilised metal affinity chromatography (IMAC) using a re-folding technique of decreasing urea concentration. At 0M urea, the SNUT-CD33 was eluted from the IMAC column and analysed by SDS PAGE using Coomassie blue stain (FIG. 7A) and Western Blotting (FIG. 7B) using anti-histag antibody.

Antibody Detection of Expressed Protein:

The SNUT fusion protein contains an N-terminal His-tag. This facilitates detection using commercially available anti-His antibodies, and can be used as a means for purification of the recombinant protein via IMAC as described (see FIG. 8a).

In addition, we have developed in-house a polyclonal antibody against SNUT and it also provides a detection and purification means, as demonstrated in FIG. 8b.

Furthermore, the inventor has developed monoclonal antibodies against SNUT which may also be used in detection and purification methods of the invention. A hybridoma producing monoclonal antibodies against SNUT was developed as follows:

4 BALB/c mice were immunised intraperitoneally with a purified SNUT recombinant protein. Seven inoculations of 50 μl of the antigen mixed with 50 μl of adjuvant were given over a ten-week time course. Test bleeds were taken at intervals and positive immunisation was confirmed by Western blot. Two days after final inoculation, the mouse spleen cells were fused with SP2 myeloma cells. The resulting hybridoma cells were maintained in HAT media. Microtitre plates were coated with the immunising antigen (50 ng/well) together with a control. Eleven days post fusion actively growing Hybridoma cells were ELISA screened for specificity to SNUT. Those giving high readings were cloned twice by limiting dilutions. An ECL of supernatant was performed as a final control of their specificity.

FIG. 8C shows a Western blot showing the detection of the SNUT protein using one of the monoclonal antibodies developed.

Results:

CD33 has been a very difficult protein to express. The most desirable part of the protein for antigen production is the extracellular variable domain. There are two immunoglobulin domains in the extracellular region of CD33, a membrane distal variable (IgV) domain and a membrane proximal constant (C2) domain. Expression analysis had been performed for three fragments of the extracellular region: the variable domain, the constant domain and the full extracellular region in a number of commercially available, expression vectors. Only the constant domain fragment would express in any of the vectors. In order to express the desired variable domain, the full length extracellular fragment and the IgV domain fragment were cloned into our pSNUT vector. Expression was successful for the full length fragment.

The full length fragment was also purified successfully by re-folding on an IMAC column. Not only has the pSNUT vector allowed us to express a protein fragment that has been unable to be expressed in any tried commercially available vector, including vectors with fusion tags designed to increase expression such as NusA and DHFR, but has allowed us to purify the expressed protein using immobilised metal affinity chromatography by standard techniques, and can be used for detection of any protein expressed in the vector using either anti-His or anti-SrtA antibodies.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. (canceled)

2. (canceled)

3. (canceled)

4. (canceled)

5. (canceled)

6. An expression construct for the production of recombinant polypeptides, which construct comprises an expression cassette consisting of the following elements that are operably linked: a) a promoter; b) the coding region of a DNA encoding a sortase gene product as a purification tag sequence; and c) a cloning site for receiving the coding region for the recombinant polypeptide to be produced; and d) transcription termination signals.

7. The expression construct according to claim 6 wherein the sortase gene product is a Staphylococcus aureus srtA gene product.

8. The expression construct according to claim 6 wherein the sortase gene product is encoded by the nucleotide sequence shown in FIG. 4 or a variant or fragment thereof.

9. The expression construct according to claim 6 wherein the sortase gene product comprises amino acids 26 to 171 of the SrtA sequence shown in FIG. 4 or a variant or fragment thereof.

10. A method for producing a polypeptide, comprising:
a) preparing an expression vector for the polypeptide to be produced by cloning the coding sequence for the polypeptide into the cloning site of an expression construct as claimed in claim 6;
b) transforming a suitable host cell with the expression construct thus obtained; and
c) culturing the host cell under conditions allowing expression of a fusion polypeptide consisting of the amino acid sequence of the purification tag with the amino acid sequence of the polypeptide to be expressed covalently linked thereto; and
d) isolating the fusion polypeptide from the host cell or the culture medium by means of binding the fusion polypeptide present therein through the amino acid sequence of the purification tag.

11. The method according to claim 10, wherein the sortase gene product is a Staphylococcus aureus srtA gene product.

12. The method according to claim 10 wherein the sortase gene product is encoded by the nucleotide sequence shown in FIG. 4 or a variant or fragment thereof.

13. The method according to claim 10 wherein the sortase gene product comprises amino acids 26 to 171 of the SrtA sequence shown in FIG. 4 or a variant or fragment thereof.

14. A fusion polypeptide obtained by the method of any one of claims 10 to 13.

15. A purification tag comprising a sortase gene product.

16. The purification tag according to claim 15 wherein the gene product is a Staphylococcus aureus srtA gene product.

17. The purification tag according to claim 15 wherein the sortase gene product is encoded by the nucleotide sequence shown in FIG. 4 or a variant or fragment thereof.

18. The purification tag according to claim 15 wherein the sortase gene product comprises amino acids 26 to 171 of the SrtA sequence shown in FIG. 4 or a variant or fragment thereof.

19. A method of inducing and/or enhancing an immune response to an antigen of interest, the method comprising administering the antigen of interest with a sortase gene product.

20. The method according to claim 19, wherein the sortase gene product is a Staphylococcus aureus srtA gene product.

21. The method according to claim 19 wherein the sortase gene product is encoded by the nucleotide sequence shown in FIG. 4 or a variant or fragment thereof.

22. The method according to claim 19 wherein the sortase gene product comprises amino acids 26 to 171 of the SrtA sequence shown in FIG. 4 or a variant or fragment thereof.
